(19) United States
(12) Patent Application Publication
(10) Pub. No.: US 2023/0371558 A1
(43) Pub. Date: Nov. 23, 2023

(54) REFRIGERATOR HAVING STERILIZATION FUNCTION

(71) Applicant: Samsung Electronics Co.,Ltd., Suwon-si (KR)

(72) Inventors: Jihee An, Suwon-si (KR); Chohui Kim, Suwon-si (KR); Jeongrok Oh, Suwon-si (KR); Sungwoo Choi, Suwon-si (KR); Chulsoo Yoon, Suwon-si (KR)

(73) Assignee: Samsung Electronics Co., Ltd., Suwon-si (KR)

(21) Appl. No.: 18/200,956

(22) Filed: May 23, 2023

(30) Foreign Application Priority Data
May 23, 2022 (KR) 10-2022-0062994

(51) Int. Cl. A23L 3/26; F25D 29/00; A23L 3/00; A61L 2/24; A61L 2/26; A61L 2/08

(52) U.S. Cl. CPC A23L 3/26; F25D 29/00; A23L 3/001; A23L 3/003; A61L 2/24; A61L 2/26; A61L 2/08; F25D 2700/12; F25D 2700/02; F25D 2700/06; A61L 2202/11; A61L 2202/14

(57) ABSTRACT

A refrigerator includes a body including a storage compartment; a door configured to open and close the storage compartment; a door opening and closing sensor configured to detect a door open state and a door closed state; a temperature sensor provided in the storage compartment, and configured to detect a temperature of the storage compartment; an illuminator provided in the storage compartment and configured to irradiate an item in the storage compartment with the light; a storage unit configured to store sterilization information including at least one preset dose for sterilization of the item; and a processor configured to: drive the illuminator to irradiate the item with the light at the preset dose, based on a signal output by the door opening and closing sensor indicating that the door is in the door closed state and the measured temperature being outside of a preset temperature range, and terminate driving of the illuminator, based on an irradiation time period for the preset dose elapsing.

### CROSS-REFERENCE TO RELATED APPLICATION(S)

This application is based on and claims priority to Korean Patent Application No. 10-2022-0062994, filed on May 23, 2022 in the Korean Intellectual Property Office, the disclosure of which is incorporated herein by reference in its entirety.

### BACKGROUND

The present disclosure relates to a refrigerator with a sterilization function.

In general, a refrigerator (e.g., a home appliance storing foods at low temperature in an internal space shielded by a door) cools the inside of the storage compartment using cold air generated through heat exchange with a refrigerant circulating in a refrigeration cycle, thereby allowing foods to be stored for a relatively long time.

Various foods may be properly stored in accommodating portions such as shelves, drawers, and baskets provided in the storage compartment of the refrigerator, and various methods have been researched/developed to provide optimal storage conditions according to types of items. For example, storage may be optimized by independently controlling temperature and/or humidity according to a type of an item stored in an accommodating portion.

A refrigerator may be configured to store an item (for example, food) at low temperatures using a refrigeration cycle. Such a refrigerator may be required to have a sterilization function to enhance freshness. However, ultraviolet (UV) light mainly used for sterilization lighting is harmful to a human body (skin or eyes), and the use thereof may be limited. In addition, when excessively exposed to sterilization lighting, there may be an issue of discoloration or degradation in quality depending on a type of item, which requires appropriate control according to a type and a storage state of a stored item.

### SUMMARY

One or more example embodiments provide a refrigerator having a sterilization function which may improve the freshness of an item.

Additional aspects will be set forth in part in the description which follows and, in part, will be apparent from the description, or may be learned by practice of the presented embodiments.

According to an aspect of an example embodiment, a refrigerator includes: a body including a storage compartment; a door provided on the body and configured to open and close the storage compartment; a door opening and closing sensor configured to detect a door open state and a door closed state; a temperature sensor provided in the storage compartment, and configured to detect a temperature of the storage compartment; an illuminator provided in the storage compartment and including a light-emitting diode (LED) light source configured to emit light having a wavelength of 380 nm to 420 nm, the illuminator being configured to irradiate an item in the storage compartment with the light; a storage unit configured to store sterilization information including at least one preset dose for sterilization of the item; and a processor configured to: drive the illuminator to irradiate the item in the storage compartment with the light at the preset dose, based on a signal output by the door opening and closing sensor indicating that the door is in the door closed state and the temperature of the storage compartment measured by the temperature sensor being outside of a preset temperature range, and terminate driving of the illuminator, based on an irradiation time period for the preset dose elapsing.

According to an aspect of an example embodiment, a refrigerator includes: a body including a storage compartment in which a plurality of accommodating portions are provided; a door provided on the body and configured to open and close the storage compartment; a door opening and closing sensor configured to detect whether the door is open or closed; a temperature sensor provided in the storage compartment, and configured to detect a temperature of the storage compartment; a plurality of illuminators respectively provided in the plurality of accommodating portions, wherein each of the plurality of illuminators includes a light-emitting diode (LED) light source configured to emit light having a wavelength of about 380 nm to about 420 nm, and the plurality of illuminators are configured to irradiate items accommodated in the plurality of accommodating portions with the light; a storage unit configured to store dosage information based on a type of an item, the dosage information including a first dose at a lower limit, and a second dose at an upper limit, the first dose and the second dose selected from a range of about 0.1 to about 200 J/cm2; an input unit configured to select a type of each item of the items accommodated in the plurality of accommodating portions based on an input of a user; and a processor configured to independently drive the plurality of illuminators so as to irradiate the items in the plurality of accommodating portions with the light based the dosage information corresponding to the type of each item selected by the input unit, based on a signal output by the door opening and closing sensor indicating that the door is closed and the temperature of the storage compartment measured by the temperature sensor being outside of a preset temperature range.

According to an aspect of an example embodiment, a refrigerator includes: a body including a storage compartment in which a plurality of accommodating portions are provided; a door provided on the body and configured to open and close the storage compartment; a door opening/closing sensor configured to detect whether the door is open or closed; a temperature sensor provided in the storage compartment, and configured to detect a temperature of the storage compartment; a plurality of illuminators respectively provided in the plurality of accommodating portions, wherein each of the plurality of illuminators includes a light-emitting diode (LED) light source configured to emit light having a wavelength of about 380 nm to about 420 nm, and the plurality of illuminators are configured to irradiate items accommodated in the plurality of accommodating portions with the light; a distance measurement sensor or a weight sensor configured to determine a distance between an item in at least one of the plurality of accommodating portions and an LED light source of at least one of the plurality of illuminators; a storage unit configured to store sterilization information including at least one preset dose for sterilization based on a type of the item; and a processor configured to: drive the at least one of the plurality of illuminators so as to irradiate the item accommodated in the at least one of the plurality of accommodating portions with the light at the preset dose, based on a signal output by the door opening and closing sensor indicating that the door is closed and the temperature of the storage compartment measured by the temperature sensor being outside of a preset temperature range, control a current applied to the at least one of the plurality of illuminators, based on the determined distance, and terminate driving of the at least one of the plurality of illuminators, based on an irradiation time period for the preset dose elapsing.

The various and beneficial advantages and effects of example embodiments are not limited to the above description, and will be more easily understood in the course of describing specific example embodiments.

### BRIEF DESCRIPTION OF DRAWINGS

The above and other aspects, features, and advantages of certain example embodiments of the present disclosure will be more apparent from the following description taken in conjunction with the accompanying drawings, in which:

FIG. 1A is a diagram illustrating a front surface of a refrigerator in a door closed state according to an example embodiment;

FIG. 1B is a diagram illustrating a front surface of a refrigerator in a door open state according to an example embodiment;

FIG. 2 is a diagram illustrating an illuminator and an accommodating portion disposed in a storage compartment of a refrigerator according to an example embodiment;

FIG. 3 is a diagram illustrating an interior of an accommodating portion according to an example embodiment;

FIG. 4 is a diagram illustrating a configuration for a sterilization operation of a refrigerator according to an example embodiment;

FIG. 5 is a diagram illustrating an illuminator according to an example embodiment;

FIG. 6 is a cross-sectional view illustrating a light-emitting diode (LED) package employable in an illuminator according to an example embodiment;

FIG. 7 is a flowchart illustrating a sterilization operation of a refrigerator according to an example embodiment;

FIG. 8 is a time graph illustrating a sterilization operation according to an example embodiment;

FIG. 9 is a time graph illustrating a sterilization operation according to another example embodiment;

FIG. 10 is a diagram illustrating a configuration for a sterilization operation of a refrigerator according to an example embodiment; and

FIG. 11 is a diagram illustrating an illuminator according to an example embodiment.

### DETAILED DESCRIPTION

Hereinafter, various example embodiments will be described in detail with reference to the accompanying drawings.

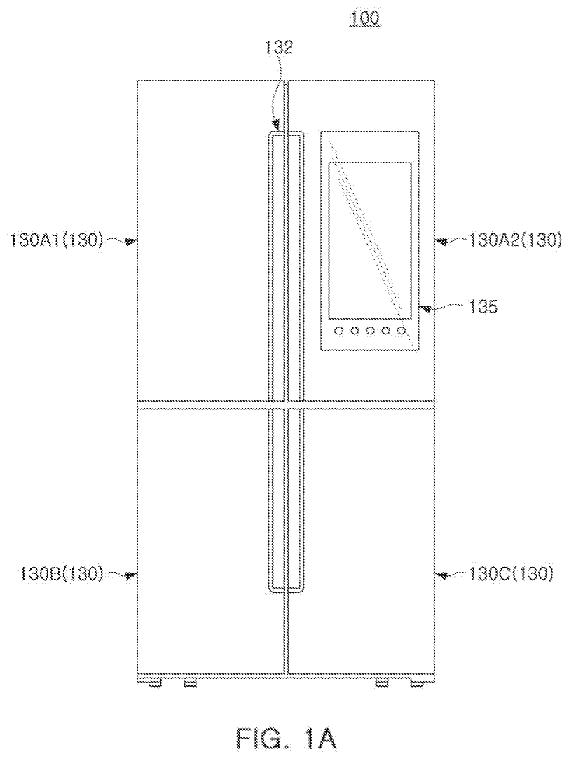

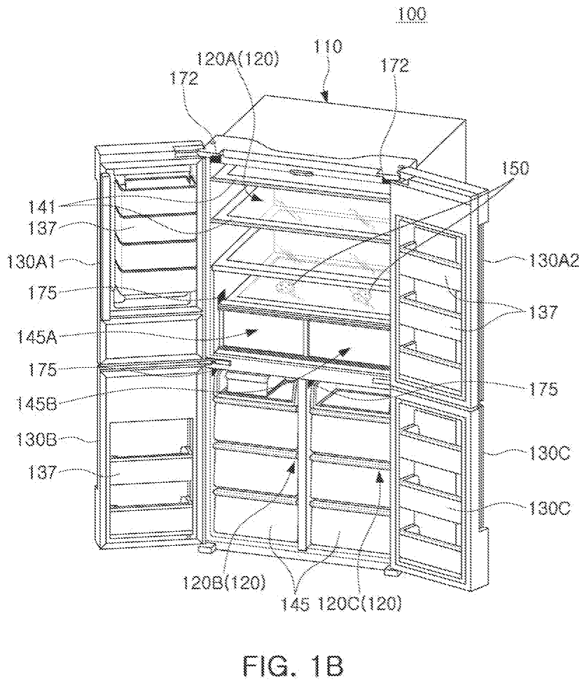

FIG. 1A is a diagram illustrating a front surface of a refrigerator in a door closed state according to an example embodiment. FIG. 1B is a diagram illustrating a front surface of a refrigerator in a door open state according to an example embodiment.

Referring to FIGS. 1A and 1B, a refrigerator 100 according to example embodiments may include a body 110 having an open front surface, a storage compartment 120 for cooling and freezing an item (for example, food) in the body 110, and a door 130 configured to open and close the open front surface of the body 110.

The body 110 may provide an exterior of the refrigerator 100, and may have a structure in which an insulating material capable of preventing outflow of cold air from the storage compartment 120 is filled. The storage compartment 120 may be divided into a plurality of storage compartments 120A, 120B, and 120C by horizontal and/or vertical partition walls. For example, as illustrated in FIG. 1B, the storage compartment 120 employed in example embodiments may be divided into a first storage compartment 120A positioned on an upper portion thereof, and second and third storage compartments 120B and 120C positioned on a lower portion thereof.

A door 130 employed in example embodiments may include first and second doors 130A1 and 130A2 hingedly connected to opposite sides of an upper portion of the body 110, and third and fourth doors 130B and 130C hingedly connected to opposite sides of a lower portion of the body 110. Thus, the first and second doors 130A1 and 130A2 may be configured to open and close a first storage compartment 120A as a double door, and the third and fourth doors 130B and 130C may be configured to respectively open and close a second storage compartment 120B and a third storage compartment 120C as a single door. Example embodiments are not limited thereto, and the storage compartment 120 may be divided to have various numbers and arrangements, and the door 130 may also be arranged in various forms according to arrangement of each storage compartment 120.

The doors 130A1, 130A2, 130B and 130C may be provided with a handle 132 such that the doors 130A1, 130A2, 130B and 130C are easily open and closed. The handle 132 may be formed to be elongate in a vertical direction along a space between the first and second doors 130A1 and 130A1 and a space between the third and fourth doors 130B and 130C.

Various types of accommodating portions may be installed in the storage compartment 120. Referring to FIG. 1B, a plurality of shelves 141 on which storage items are placeable may be installed in the first storage compartment 120A.

In addition, a plurality of drawers 145 for storing various types of items may be respectively installed in the first to third storage compartments 120A, 120B, and 120C. The drawers 145 may be configured to be inserted into or removed from the storage compartment 120 according to a guide installed in the storage compartment 120. The accommodating portion of the refrigerator 100 may be provided with a door guard 137 for accommodating a small-sized food package or bottle on an inner surface of the door 130 in addition to a space provided in the storage compartment 120.

The refrigerator 100 according to example embodiments may include an illuminator 150 disposed in the storage compartment 120 and configured to perform a sterilization operation. The illuminator 150 employed in example embodiments may use, as light for sterilization, light having a visible ray band, for example, light having a wavelength of 380 to 420 nm, instead of ultraviolet light, thereby greatly reducing harmfulness to a human body.

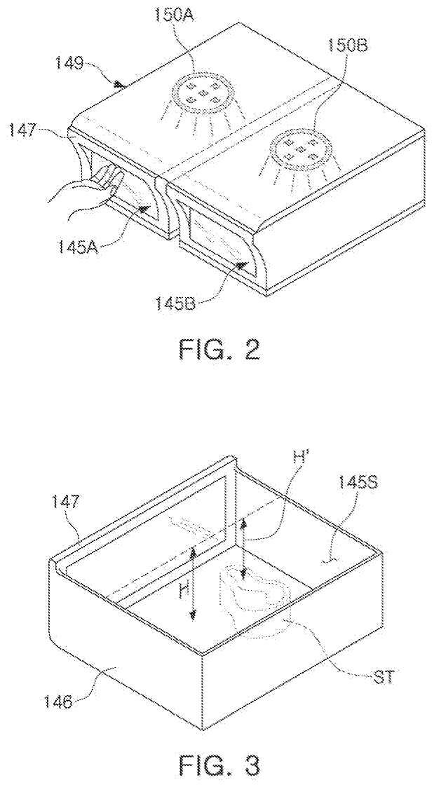

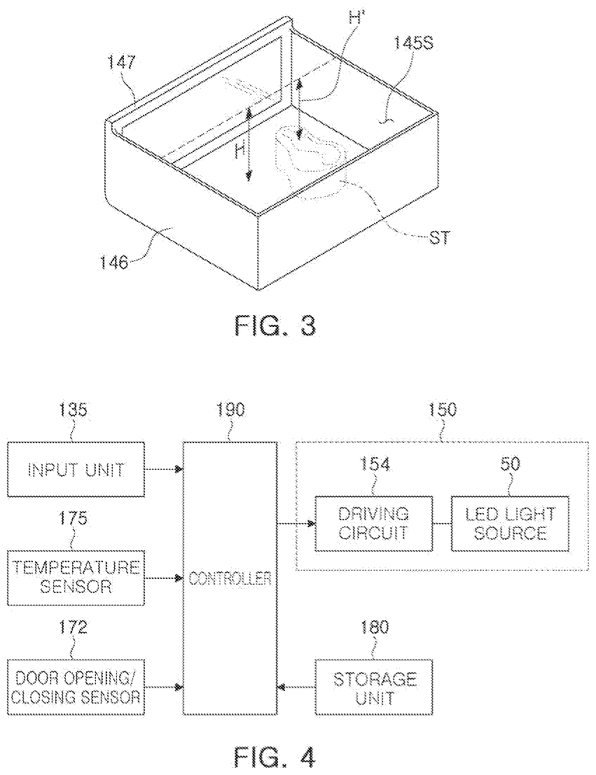

FIG. 2 is a diagram illustrating an illuminator and an accommodating portion disposed in a storage compartment of a refrigerator according to an example embodiment. FIG. 3 is a diagram illustrating an interior of an accommodating portion according to an example embodiment.

Referring to FIGS. 2 and 3, the illuminator 150 employed in example embodiments may be disposed in some drawers 145A and 145B (hereinafter referred to as “sterilization drawer”) of the plurality of drawers 145. First and second illuminators 150A and 150B may be disposed in first and second sterilization drawers 145A and 145B, respectively.

The first and second sterilization drawers 145A and 145B may respectively include a drawer case 146 having an accommodating space 145S, and the first and second illuminators 150A and 150B may be respectively disposed below a drawer upper plate 149 so as to easily irradiate an item ST to be disposed in the drawer case 146. In example embodiments, in the first and second illuminators 150A and 150B, a light guide member may be additionally installed over a significant area of the upper plate 149 to enable uniform light irradiation, despite various arrangements (for example, a storage height and a position) of the item ST. Front panels 147 of the first and second sterilization drawers 145A and 145B may be formed of a transparent material such that a user checks a state of an item in the accommodating space 145S.

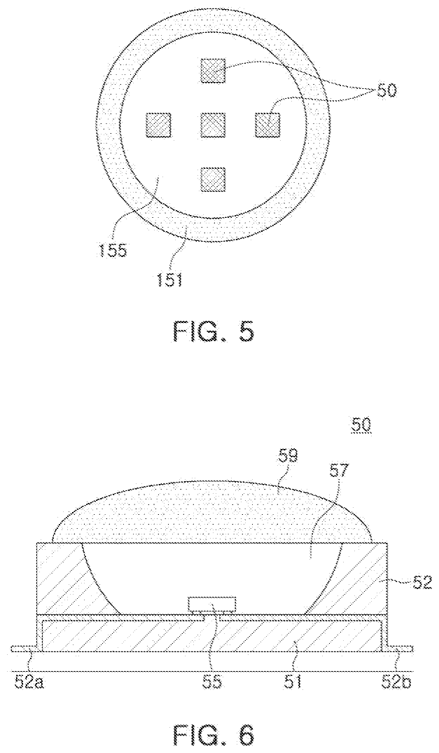

The first and second illuminators 150A and 150B employed in example embodiments may respectively include a light-emitting diode (LED) light source (“50” in FIG. 5) emitting light having a wavelength of about 380 nm to about 420 nm, and may be configured to irradiate an item stored in the first and second sterilization drawers 145A and 145B with the light.

In example embodiments, a dose necessary for sterilization of an item may be set to have a range of about 0.1 to about 200 J/cm2 in terms of sterilization and quality control, and information on the dose may be set to a dose range varying according to a type of the item. In addition, the dosage information according to the type of the item may be pre-stored in the storage unit (“180” in FIG. 4) of the refrigerator 100. In an example embodiment, depending on the usage environments and the type of the item, an appropriate dose may be selected from a range of a dose required for sterilization, and may be pre-stored in in the storage unit.

In example embodiments, a dose range necessary for sterilization may have a lower limit condition (for example, a first dose) capable of sufficiently removing harmful microorganisms such as Escherichia coli and staphylococcus, viruses and bacteria from items such as meat and vegetables. In addition, an excessive dose range may cause discoloration and quality degradation, the dose range necessary for sterilization may have an upper limit condition (for example, a second dose), based on determination on a relative color change for each type of item.

For example, when a light source having a wavelength of about 405 nm is used, a dose range for meat and vegetables may define a dose necessary for sterilization through results indicated in Tables 1 and 2 below.

TABLE 1

| Meat | Sterilization power (%) | | | Relative color change | | | |
|---|---|---|---|---|---|---|---|
| Dose | Escherichia | | General | ΔL | Δa | Δb | Discoloration |
| (J/cm²) | coli | Staphylococcus | bacteria | (brightness) | (red) | (yellow) | evaluation |
| 0.05 | 94 | 94 | 94 | 0 | 0 | 0 | Good |
| 0.1 | 99.9 | 99.9 | 99.9 | 0 | 0 | 0 | Good |
| 1 | 99.9 | 99.9 | 99.9 | 0 | 0 | 0 | Good |
| 10 | 99.9 | 99.9 | 99.9 | 0 | 0 | 0 | Good |
| 20 | 99.9 | 99.9 | 99.9 | 0 | 0 | 0 | Good |
| 40 | 99.9 | 99.9 | 99.9 | 0 | 0 | 0 | Good |
| 60 | 99.9 | 99.9 | 99.9 | 0 | 0 | 0 | Good |
| 80 | 99.9 | 99.9 | 99.9 | 0 | 0 | 0 | Good |
| 100 | 99.9 | 99.9 | 99.9 | 0 | 0 | 0 | Good |
| 120 | 99.9 | 99.9 | 99.9 | 0 | 0 | 0 | Good |
| 140 | 99.9 | 99.9 | 99.9 | 0 | −0.25 | −0.25 | Normal |
| 160 | 99.9 | 99.9 | 99.9 | 0 | −0.75 | −0.75 | Normal |
| 180 | 99.9 | 99.9 | 99.9 | 0.10 | −1.25 | −1.25 | Normal |
| 200 | 99.9 | 99.9 | 99.9 | 0.20 | −1.75 | −1.75 | Normal |
| 210 | 99.9 | 99.9 | 99.9 | 0.30 | −2.00 | −2.00 | Bad |
| 230 | 99.9 | 99.9 | 99.9 | 0.40 | −2.50 | −2.50 | Bad |

TABLE 2

| Vegetable | Sterilization power (%) | | | Relative color change | | | |
|---|---|---|---|---|---|---|---|
| Dose | Escherichia | | General | ΔL | Δa | Δb | Discoloration |
| ((J/cm²) | coli | Staphylococcus | bacteria | (brightness) | (red) | (yellow) | evaluation |
| 0.05 | 94 | 94 | 94 | 0 | 0 | 0 | Good |
| 0.1 | 99.9 | 99.9 | 99.9 | 0 | 0 | 0 | Good |
| 1 | 99.9 | 99.9 | 99.9 | 0 | 0 | 0 | Good |
| 10 | 99.9 | 99.9 | 99.9 | 0 | 0 | 0 | Good |
| 20 | 99.9 | 99.9 | 99.9 | 0 | 0 | 0 | Good |
| 40 | 99.9 | 99.9 | 99.9 | 0 | −0.25 | −0.25 | Normal |
| 60 | 99.9 | 99.9 | 99.9 | 0 | −0.75 | −0.75 | Normal |
| 80 | 99.9 | 99.9 | 99.9 | 0.10 | −1.25 | −1.25 | Normal |
| 100 | 99.9 | 99.9 | 99.9 | 0.20 | −1.75 | −1.75 | Normal |
| 120 | 99.9 | 99.9 | 99.9 | 0.30 | −2.25 | −2.25 | Bad |
| 140 | 99.9 | 99.9 | 99.9 | 0.40 | −2.75 | −2.75 | Bad |
| 150 | 99.9 | 99.9 | 99.9 | 0.50 | −3.00 | −3.00 | Bad |

Specifically, when an item is meat, sufficient sterilization power was provided when a dose was 0.05 J/cm², and a desired high sterilization power (99.9%) was not provided when the dose was at least 0.1 J/cm². A relative color change may be observed when the dose was 120 J/cm² or more, and the color change having a value of less than −2.0 was an allowable range (that is, “Normal”) when the dose was at most 200 J/cm². However, when the dose is greater than 200 J/cm², the relative color change range was increased, causing discoloration resulting in a degradation in freshness.

Accordingly, when the item is meat, a proper sterilization dose may be in a range of about 0.1 to about 200 J/cm².

In addition, when the item is a vegetable, sufficient sterilization power was not provided when the dose is 0.05 J/cm², and a desired high sterilization power (99.9%) was not provided when the dose was at least 0.1 J/cm². A relative color change may be observed when the dose was 40 J/cm² or more, and the color change having a value of less than −2.0 was an allowable range (that is, “Normal”) when the dose was at most 100 J/cm². However, when the dose is greater than 100 J/cm², the relative color change range was increased, causing discoloration resulting in a degradation in freshness.

Accordingly, when the item is a vegetable, a proper sterilization dose may be in a range of about 0.1 to about 100 J/cm².

In a similar manner, in the case of food stored at room temperature, a sterilization dose may be set to have a range of about 0.1 to about 50 J/cm².

Thus, in the above-described range in which a minimum dose (e.g., a first dose) necessary for sterilization and a maximum dose (e.g., second dose) for preventing discoloration and quality degradation are defined according to a type of each item, a desired dose range (e.g., first and second doses) may be selected and set (e.g., input by a user or pre-stored in a storage unit) according to the type of each item and a storage environment, for sterilization of an actual refrigerator.

In some example embodiments, the first dose and the second dose may be respectively selected and set as specific values in the above-described range regardless of a type of an item. For example, the first dose may be set to 0.05 J/cm² or more, and the second dose may be set to 210 J/cm² or less.

In addition, as illustrated in FIG. 2, a sterilization operation using each illuminator 150A may be independently performed on an item, stored in the first and second sterilization drawers 145A and 145B, at a dose necessary for a type of the item. The first and second doses may be set in various manners, as indicated in Table 3 below, under a condition satisfying the above-described dose range.

TABLE 3

| | First dose | Second | |
|---|---|---|---|
| Classification | (J/cm²) | dose (J/cm²) | Remark |
| Example 1 | 0.1 | 10 | Deep compartment, and |
| | | | meat/vegetable/room |
| | | | temperature |
| Example 2 | 30 | 50 | Deep compartment, and |
| | | | meat/vegetable/room |
| | | | temperature |
| Example 3 | 0.1 | 20 | Deep compartment, and |
| | | | meat/vegetable |
| Example 4 | 10 | 100 | Deep compartment, and |
| | | | meat/vegetable |

In addition, as described in the Remark column, a set value regardless of a type of an item may be set according to an accommodating portion structure (in particular, a depth) and an accommodating portion to which an item to be stored is designated. A depth of the accommodating portion refers to a distance H to a bottom surface of the drawer case 146, as illustrated in FIG. 3. For example, a dose may be set in a relatively low range to prevent discoloration caused by an excessive dose, since the distance between the item and the LED light source varies greatly depending on the location of the item in an accommodating portion having a significant depth.

FIG. 4 is a diagram illustrating a configuration for a sterilization operation of a refrigerator according to an example embodiment.

Hereinafter, configurations for the sterilization operation of the refrigerator 100 according to example embodiments will be described in detail with reference to FIG. 4 together with FIGS. 1A and 1B.

As illustrated in FIG. 1A, an input unit 135 may be provided on a front surface of a door 130B. The input unit 135 may be a button for a user to input an operation signal (for example, a set temperature, or the like) of the refrigerator 100 and a display input unit displaying an operation state of a refrigerator. For example, the input unit 135 may include a touch screen display. The touch screen display may include a display panel displaying an image, a touch panel receiving a touch input, and a touch screen controller driving/controlling the display panel and the touch panel. The display panel may convert image information received through the touch screen controller into an optical signal that is viewable by a user.

The operation signal selected by the user through the input unit 135 may be transmitted to a controller 190 controlling an overall operation of the refrigerator 100.

Specifically, as indicated in Table 4 below, a first dose and a second dose for setting a dose necessary for sterilization may be automatically set by predetermined values according to a type of an item selected by the user who operates the refrigerator.

TABLE 4

| | First dose | Second dose |
|---|---|---|
| Item type | (J/cm²) | (J/cm²) |
| Food stored at room | 0.1 | 10 |
| temperature | | |
| Vegetable | 0.5 | 30 |
| Meat | 10 | 100 |

Alternatively, when a plurality of accommodating portions for sterilization are included, the first dose and the second dose may be set for each accommodating portion, as illustrated in Table 5 below.

TABLE 5

| | First dose | Second dose |
|---|---|---|
| Accommodating portion | (J/cm²) | (J/cm²) |
| Accommodating portion for | 0.1 | 10 |
| room-temperature storage | | |
| Vegetable accommodating portion | 0.5 | 30 |
| Meat accommodating portion | 10 | 100 |

The controller 190 may be configured to control an overall operation of the refrigerator such as a sterilization operation of an illuminator, in addition to a configuring device such as a compressor forming a cooling cycle, and a cooling air supply device, or to perform various control operations. For example, the controller 190 may include a processor or microprocessor having a central processing unit (CPU), a microcontroller, or the like.

In addition, the refrigerator 100 may include a storage unit 180 storing information necessary for the operation of the controller 190. For example, the storage unit 180 may include a memory device such as a random access memory (RAM), a read only memory (ROM), or a flash memory. In example embodiments, the storage unit 180 may be implemented as a module integrated with a processor included in the controller 190.

For the sterilization operation according to example embodiments, the input unit 135 may be configured to select a type of an item to be sterilized by a user input. In example embodiments, when the user stores a desired item in each of the first and second sterilization drawers 145A and 145B and the user selects a type of the stored item through the input unit 135, the controller 190 may receive, from the storage unit 180, dosage information necessary for sterilization according to the type of the selected item to independently perform the sterilization operation on the item stored in each of the first and second sterilization drawers 145A and 145B through the first and second illuminators 150A and 150B.

The refrigerator 100 according to example embodiments may further include a door opening and closing (opening/closing) sensor 172 and a temperature sensor 175.

As illustrated in FIG. 1B, the door opening/closing sensor 172 may be configured to detect whether the door 130 is open or closed (or in an open state or in a closed state) by selectively contacting upper both sides of the body 110 by means of an opening/closing operation of the door 130. The door opening/closing sensor 172 may be electrically connected to the controller 190, and a signal detected by the door opening/closing sensor 172 may be transmitted to the controller 190.

Similarly, the temperature sensor 175 may also be provided on a side of the storage compartment 120A to detect an internal temperature of the storage compartment 120A. The temperature sensor 175 may convert the detected temperature into an electrical signal and transmit the electrical signal to the controller 190 electrically connected to the temperature sensor 175. In example embodiments, the temperature sensor 175 may be disposed in each of the first and second sterilization drawers 145A and 145B to more accurately measure storage temperature of an item to be sterilized.

The controller 190 may compare a set temperature input through the input unit 135 with a temperature detected by the temperature sensor 175, and may drive a cold air circulation fan so as to supply cold air to the storage compartment 120 according to the set temperature.

In example embodiments, the controller 190 may start the sterilization operation of the illuminator 150 when it is determined that a door is closed and/or the set temperature exceeds a predetermined range. Specifically, when it is determined that the door is closed from a detection signal of the door opening/closing sensor 172 and/or a temperature measured by the temperature sensor 175 is outside of a preset temperature range, the controller 190 may drive the illuminator to start the sterilization operation.

In example embodiments, when the temperature of the storage compartment 120A measured by the temperature sensor 175 is higher than a preset storage compartment temperature by about 2° C. or more, it may be determined that the temperature is outside of the preset temperature range.

A driving circuit 154 of the illuminator 150 may be electrically connected to the controller 190. Depending on whether the door 130 is open and whether storage compartment temperature exceeds a set temperature range, the controller 190 may transmit an operation signal (applied current) to a driving circuit 154 of the illuminator 150 to drive the LED light source 50, thereby irradiating an item with light necessary for sterilization.

As described above, a dose necessary for sterilization may be determined differently according to a type of the item, and the controller 190 may receive, from the storage unit 180, sterilization information related to a necessary dose during the sterilization operation to drive the illuminator 150 according to the sterilization information.

The sterilization information may include an irradiation time period together with output per unit area (mW/cm²) of the LED light source 50, and the output per unit area of the LED light source 50 may be determined by applied current. Referring to Table 6 below, an irradiation time period according to applied current (output) for an item that is meat may be exemplified, and a dose necessary for sterilization may be provided to the controller 190 as a condition satisfying “Good” or “Normal.”

TABLE 6

| Applied | | | |
|---|---|---|---|
| current/Time period | 2 h | 4 h | 6 h |
| 5 mA | Good | Good | Good |
| 100 mA | Good | Good | Normal |
| 150 mA | Good | Normal | Normal |
| 200 mA | Bad | Bad | Bad |

Accordingly, in example embodiments, when an irradiation time period for the dose elapses, driving of the illuminator may be terminated to prevent excessive irradiation resulting in “Bad” such as discoloration or the like.

Such doses may vary according to a distance between the LED light source 50 and an item, in addition to output and an irradiation time period according to an applied current condition. Accordingly, in order to more effectively prevent a defect such as discoloration or the like caused by an excessive dose, such a distance condition may be considered.

Referring to FIG. 3, when a distance from a position of the LED light source 50 to a bottom surface of the drawer case 146 is referred to as “H,” a distance H′ to the item ST according to an amount and position of the item ST may be shorter. In particular, the output per unit area may be greatly affected by a distance to an item.

Referring to Table 7 below, for 50 mW/cm², an output per unit area according to each applied current and a height (that is, an amount) of the item ST, and time information necessary therefor may be presented. As the height of the item ST increases, it may be determined that a distance H′ between the item ST and the LED light source 50 decreases.

As illustrated in Table 7 below, in order to obtain the same dose (50 mW/cm²), even if applied current is the same, the output per unit area may be reduced as a height (e.g., an amount) of an item increases. Thus, an irradiation time period may be set to be longer, or the applied current may be increased.

TABLE 7

| Applied | Height | Output per unit | Time period |
|---|---|---|---|
| current (mA) | (cm) | area (mW/cm²) | (h) |
| 50 | 5 | 1.07 | 1.3 |
| | 10 | 0.28 | 5 |
| | 15 | 0.13 | 11 |
| | 20 | 0.07 | 20 |
| 100 | 5 | 2.31 | 0.6 |
| | 10 | 0.56 | 2.5 |
| | 15 | 0.25 | 5.5 |
| | 20 | 0.14 | 10 |
| 150 | 5 | 3.47 | 0.4 |
| | 10 | 0.852 | 1.7 |
| | 15 | 0.38 | 3.7 |
| | 20 | 0.21 | 6.6 |
| 200 | 5 | 4.63 | 0.3 |
| | 10 | 1.16 | 1.2 |
| | 15 | 0.50 | 2.8 |
| | 20 | 0.28 | 5 |

Thus, in order to accurately determine a dose necessary for sterilization of an item, a height of the item, that is, information on a distance to the LED light source 50 may be required. Such information may be determined using a distance measurement sensor or a weight sensor. For example, the distance measurement sensor may be mounted in the illuminator 150 or in a region of the drawer upper plate 149 on which the illuminator 150 is mounted. Alternatively, a weight of an item stored in the first and second sterilization drawers 145A and 145B may be measured instead of the distance measurement sensor, thereby calculating a volume thereof according to a type of the item, and estimating a height of the item based on the calculated volume.

In consideration of the height of the item, that is, information on the distance to the LED light source 50, the controller 190 may determine applied current and an irradiation time period for a dose of the item, and control the illuminator 150.

FIG. 5 is a diagram illustrating an illuminator according to an example embodiment. FIG. 6 is a cross-sectional view illustrating an LED package employable in an illuminator according to an example embodiment.

Referring to FIG. 5, the illuminator 150 may include a circuit board 155 on which a driving circuit is implemented, and a plurality of LED light sources 50 arranged on the circuit board 155. For example, the circuit board 155 may include a printed circuit board such as FR-4.

The illuminator 150 may include a support 151 having a surface on which the circuit board 155 is mounted, and may be fixed to the drawer upper plate (“149” in FIG. 2) using the support 151.

The plurality of LED light sources 50 may be configured to emit light having a wavelength of about 380 nm to about 420 nm. The plurality of LED light sources 50 may be exemplified on the circular circuit board 155 in a vertical/horizontal symmetric arrangement. In example embodiments, the circuit board 155 may have various other shape (e.g., rectangle) and the LED light source 50 may have various arrangements. For example, the circuit board 155 may have a bar structure arranged along edges of a drawer upper plate, and the plurality of LED light sources 50 may be arranged in a longitudinal direction of a bar. The LED light sources 50 may be provided as the LED package illustrated in FIG. 6.

Referring to FIG. 6, the LED light source 50 may include a package substrate 51, an LED chip 55 disposed on the package substrate 51, and a sidewall reflector 52 disposed on the package substrate 51 to surround the LED chip 55.

The LED light source 50 may include a pair of lead frames 52a and 52b electrically connected to the LED chip 55. In example embodiments, a form in which the LED chip 55 is connected to the lead frames 52a and 52b in a flip-chip manner is exemplified. However, in example embodiments, the LED chip 55 may be connected to the lead frames 52a and 52b by a wire.

In example embodiments, the package substrate 51 may include a ceramic substrate having superior heat dissipation performance to ensure high output. For example, the ceramic substrate may include MN or Al₂O₃. In another example embodiment, the package substrate 51 may include a resin containing highly reflective powder. The highly reflective powder may be white powder such as titanium dioxide (TiO₂).

The sidewall reflector 52 may be disposed on the package substrate 51 and the lead frames 52a and 52b and form a cavity for accommodating the LED chip 55. The sidewall reflector 52 may have a cup shape having an inclined inner sidewall to improve reflection efficiency.

A resin encapsulant 57 may be formed in the cavity to cover the LED chip 55. Light having a desired wavelength necessary for sterilization may be directly provided by an LED chip, the resin encapsulant may not include a phosphor. The resin encapsulant 57 may be formed of a light-transmissive resin, and may include, for example, epoxy, silicone, modified silicone, urethane resin, oxetane resin, acryl, polycarbonate, or polyimide. In example embodiments, the resin encapsulant 57 may include a small amount of green phosphor to provide visual comfort.

The LED light source 50 may further include a lens 59 disposed on the sidewall reflector 52 to cover an LED chip. The lens 59 may adjust an orientation angle to improve output of the LED chip 55 and improve uniformity of light in a storage compartment. For example, the lens 59 may be formed of glass or silicon.

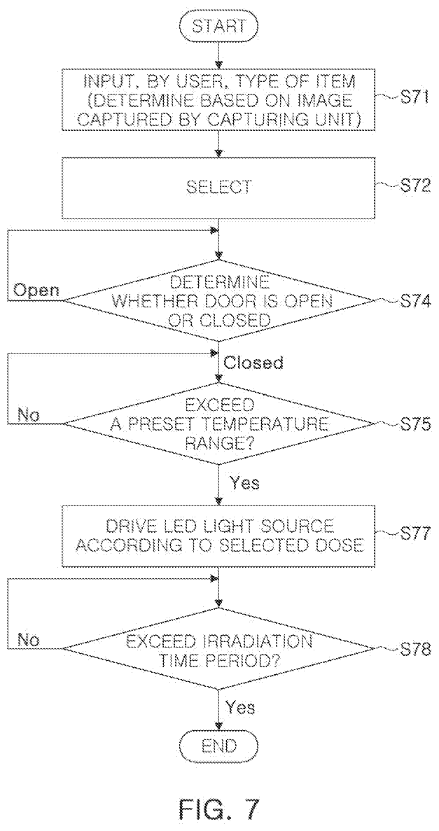

FIG. 7 is a flowchart illustrating a sterilization operation of a refrigerator according to an example embodiment.

Referring to FIG. 7, the sterilization operation according to example embodiments may be started by inputting a type of an item in operation S71.

After a user stores an item in an accommodating space for sterilization (for example, a sterilization drawer), the user may select a type of the item in the designated accommodating space through the input unit. For example, when meat is stored in a first sterilization drawer and a vegetable is stored in a second sterilization drawer, meat and vegetable may be selected from among item types in input boxes of respective sterilization drawers through a display input unit positioned on a front surface of a door.

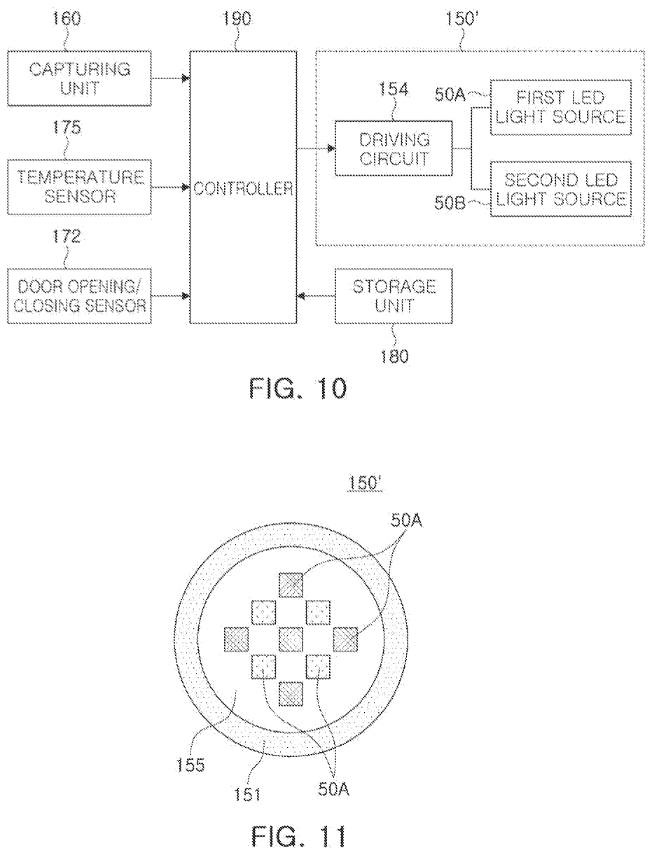

Alternatively, in example embodiments, a capturing unit may be mounted in an accommodating space for sterilization instead of a user input, and a type of a stored item may be automatically selected based on color or shape information of the item (see FIG. 10).

In operation S72, a dose necessary for sterilization may be determined based on the input type of the item.

The dose according to the type of the item may be pre-stored in the storage unit described above, and the dose may be provided to a controller controlling driving of an illuminator, based on information on light output and an irradiation time period. In example embodiments, information related to a height of the item may be additionally provided by a distance measurement sensor and a weight sensor, and the light output (or applied current) and the irradiation time period may be adjusted according to the height of the stored item.

Then, light irradiation for sterilization may be started based on determination on opening and closing of the door and a temperature change.

In operation S74, it may be determined whether the door is open or closed through the door opening/closing sensor. In a door open state (“Open” in operation S74), the illuminator may not be driven and operation S74 may be re-performed after, for example, a predetermined time period. However, in a door closed state (“Closed” in operation S74), in operation S75, it may be determined whether temperature of a storage compartment measured by a temperature sensor exceeds a set temperature range. When the measured temperature exceeds the set temperature range (“Yes” in operation S75), in operation S77, the illuminator (e.g., an LED light source) may be driven according to a dose condition selected from in operation S75. When the measured temperature does not exceed the set temperature range (“No” in operation S75), operation S75 may be re-performed, after, for example, a predetermined time period.

In order to prevent discoloration and quality degradation of the item caused by an excessive dose, in operation S78, when the dose is achieved, that is, when the irradiation time period elapses (“Yes” in operation S78), the LED driving may be stopped to terminate the sterilization operation. When the irradiation time period does not elapse (“No” in operation S78), operation 78 may continue to be performed.

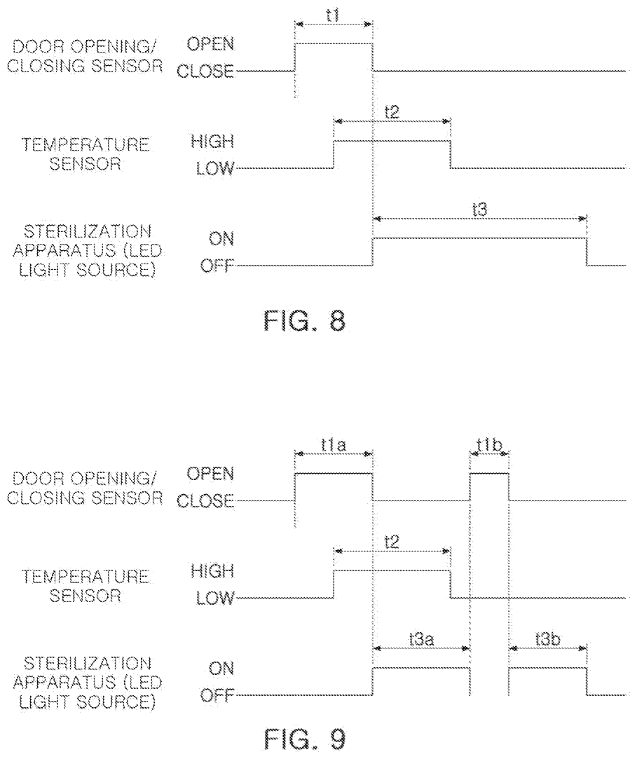

FIG. 8 is a time graph illustrating a sterilization operation according to an example embodiment.

Referring to FIG. 8, as a result of the door open state (OPEN) being maintained for a first time period (t1) by a door opening/closing sensor, it may be confirmed through a temperature sensor that temperature of a storage compartment exceeds a set temperature range due to an increase in temperature. The increase in the temperature of the storage compartment may continue for a second time period (t2) even in a door closed state (CLOSE).

Thus, when it is determined that the door is closed and/or the measured temperature exceeds the set temperature range, an LED light source may be driven by a controller for a third time period t3, and a sterilization operation for a desired dose may be started.

Even if a door closed state occurs after a door open state, when the time period (t1) of the door open state is short, the temperature of the storage compartment may not exceed the set temperature range. In this case, the sterilization operation may not be performed.

FIG. 9 is a time graph illustrating a sterilization operation according to another example embodiment.

Referring to FIG. 9, similarly to the previous example embodiment, as a result of the door open state (OPEN) being maintained for a first time period (t1a) by a door opening/closing sensor. The door open state (OPEN) may be checked by whether a temperature sensor that temperature of a storage compartment exceeds a set temperature range. The increase in the temperature of the storage compartment may continue for a second time period (t2) even in a door closed state (CLOSE).

Thus, when it is determined that the door is closed and/or the measured temperature exceeds the set temperature range, an LED light source may be set to be driven for a third time period (t3a+t3b) so as to obtain a dose necessary for a type of an item.

However, before a desired irradiation time period continues, that is, after irradiation is performed only for a partial time period (t3a), a door open state may be additionally generated for a predetermined time period (t1b). In this case, driving of the LED light source may be stopped in example embodiments.

When the storage compartment temperature does not exceed the set temperature range due to the door open state (OPEN), if the door closed state occurs again, irradiation of the LED light source may be resume for a remaining irradiation time period (t3b). In some example embodiments, according to a duration of the door open state and whether the temperature is increased, sterilization may be performed for an irradiation time period longer than the remaining irradiation time period (t3b).

In example embodiments, the LED light source may not be significantly harmful to a human body, unlike general ultraviolet light. Thus, even if a door open state occurs during the sterilization operation (LED driving), the sterilization operation may continue for a predetermined irradiation time period.

FIG. 10 is a diagram illustrating a configuration for a sterilization operation of a refrigerator according to an example embodiment.

Referring to FIG. 10, it may be understood that a sterilization system of a refrigerator according to example embodiments is similar to the sterilization system illustrated in FIG. 4, except that a type of an item is inputted by a capturing unit 160 instead of a user input through an input unit, and the illuminator 150 may include a second LED light source 50B for lighting in addition to a first LED light source 50A for sterilization. In addition, components of example embodiments may be understood with reference to the descriptions of the same or similar components of the refrigerator and the sterilization system illustrated in FIGS. 1 to 4, unless otherwise specified.

The sterilization system according to example embodiments may include the capturing unit 160 mounted in an accommodating space for sterilization instead of a user input. For example, the capturing unit may include a charge-coupled device (CCD) imaging device. The controller 190 may analyze an image captured by the capturing unit 160 to estimate a type of a stored item based on color or shape information of the stored item. Based on the estimated information, an illuminator 150′ may be driven by receiving sterilization information (that is, dose) of the item from a storage unit.

The illuminator 150′ employed in example embodiments may include a second LED light source 50B for lighting in addition to the first LED light source 50A for sterilization. The first LED light source 50A may be configured to emit light having a wavelength of 380 nm to 420 nm, and the second LED light source 50B may be configured to emit colored or white light. The second LED light source 50B may be white light having a low color temperature.

FIG. 11 is a diagram illustrating an illuminator according to an example embodiment. Referring to FIG. 11, the illuminator 150′ employed in example embodiments may include a plurality of first LED light sources 50A and a plurality of second LED light sources 50B mounted on the same circuit board 155. A driving circuit of the circuit board 155 may be configured such that the plurality of first LED light sources 50A and the plurality of second LED light sources 50B are driven independently of each other. When it is determined that a door is open from a detection signal of a door opening/closing sensor, driving of the first LED light source 50A may be stopped, and the second LED light source 50B may be driven. However, example embodiments are not limited thereto. The first LED light source 50A may also have a visible light band with significantly low harmfulness, and thus may be configured to be continuously driven even if a door open state occurs for an irradiation time period for a sterilization operation.

According to example embodiments, driving of sterilization may be started according to the opening and closing of a door and a temperature condition, while maintaining the freshness of an item (for example, food) using light having a wavelength band for bacteria sterilization. In addition, a sterilization time period (that is, an irradiation time period) may be controlled such that a dose varies according to a type of the item, thereby preventing discoloration or quality degradation of the item caused by an excessive dose.

According to example embodiments, a method of increasing the freshness of an item stored in refrigeration or at room temperature using an LED light source of about 380 nm to about 420 nm in wavelength having a bacterial sterilization effect is provided. Depending on a type of item, a dose required for sterilization may be set from within a range of 0.1-200 J/cm². Such a setting may be performed by a user input or an image analysis of a capturing unit. The dose of the LED light source may be controlled based on an output of the LED light source, an irradiation time period, and a distance to the item.

While example embodiments have been shown and described above, it will be apparent to those skilled in the art that modifications and variations could be made without departing from the scope of example embodiments as defined by the appended claims.

What is claimed is:

1. A refrigerator comprising:
a body comprising a storage compartment;
a door provided on the body and configured to open and close the storage compartment;
a door opening and closing sensor configured to detect a door open state and a door closed state;
a temperature sensor provided in the storage compartment, and configured to detect a temperature of the storage compartment;
an illuminator provided in the storage compartment and comprising a light-emitting diode (LED) light source configured to emit light having a wavelength of 380 nm to 420 nm, the illuminator being configured to irradiate an item in the storage compartment with the light;
a storage unit configured to store sterilization information comprising at least one preset dose for sterilization of the item; and
a processor configured to:
drive the illuminator to irradiate the item in the storage compartment with the light at the preset dose, based on a signal output by the door opening and closing sensor indicating that the door is in the door closed state and the temperature of the storage compartment measured by the temperature sensor being outside of a preset temperature range, and
terminate driving of the illuminator, based on an irradiation time period for the preset dose elapsing.

2. The refrigerator of claim 1, further comprising:
an input unit configured to select a type of the item to be sterilized based on an input of a user,
wherein the processor is further configured to:
output sterilization information of the storage unit based on the type of the item selected by the input unit, and
drive the illuminator based on the output sterilization information.

3. The refrigerator of claim 1, further comprising:
an image capturing unit configured to capture an image of an item stored in the storage compartment,
wherein the processor is further configured to
identify, based on the image captured by the image capturing unit, a type of the item,
output sterilization information of the storage unit based on the type of the item, and
drive the illuminator based on the output sterilization information.

4. The refrigerator of claim 1, wherein the processor is further configured to determine that the temperature measured by the temperature sensor is outside of the preset temperature range, based on the temperature measured by the temperature sensor being higher than a preset temperature by 2° C.

5. The refrigerator of claim 1, wherein the processor is further configured to stop driving of the illuminator, based on the door open state occurring while driving the illuminator.

6. The refrigerator of claim 5, wherein the processor is further configured to drive the illuminator for a remaining irradiation time period based on the door closed state occurring after the door open state occurring.

7. The refrigerator of claim 1, wherein the sterilization information comprises an irradiation time period based on an output (mW/cm²) per unit area of the LED light source.

8. The refrigerator of claim 1, wherein the at least one preset dose for sterilization is based on a type of the item, and
wherein the at least one preset dose is selected from a range of about 0.1 to about 200 J/cm².

9. The refrigerator of claim 8, wherein the at least one preset dose is selected from a range of about 0.1 to about 100 J/cm², based on the type of the item being a vegetable.

10. The refrigerator of claim 8, wherein the at least one preset dose is selected from the range of about 0.1 to about 200 J/cm², based on the type of the item being meat.

11. The refrigerator of claim 1, wherein the storage compartment comprises a plurality of accommodating portions respectively having an independent storage space, and
wherein the illuminator is provided below an upper plate of at least one accommodating portion of the plurality of accommodating portions so as to irradiate an item in the at least one accommodating portion.

12. The refrigerator of claim 11, further comprising:
a distance sensor or a weight sensor configured to determine a distance between an item in the at least one accommodating portion and the LED light source,
wherein the processor is further configured to control at least one of light output and an irradiation time period of the illuminator based on the determined distance.

13. The refrigerator of claim 12, wherein the processor is further configured to control current applied to the illuminator based on the determined distance.

14. The refrigerator of claim 11, wherein the illuminator comprises an additional LED light source configured to emit colored light or white light, and
wherein the additional LED light source is configured to be driven based on the signal output by the door opening and closing sensor indicating that the door is in the door open state.

15. A refrigerator comprising:
a body comprising a storage compartment in which a plurality of accommodating portions are provided;
a door provided on the body and configured to open and close the storage compartment;
a door opening and closing sensor configured to detect whether the door is open or closed;
a temperature sensor provided in the storage compartment, and configured to detect a temperature of the storage compartment;
a plurality of illuminators respectively provided in the plurality of accommodating portions, wherein each of the plurality of illuminators comprises a light-emitting diode (LED) light source configured to emit light having a wavelength of about 380 nm to about 420 nm, and the plurality of illuminators are configured to irradiate items accommodated in the plurality of accommodating portions with the light;
a storage unit configured to store dosage information based on a type of an item, the dosage information comprising a first dose at a lower limit, and a second dose at an upper limit, the first dose and the second dose selected from a range of about 0.1 to about 200 J/cm²;
an input unit configured to select a type of each item of the items accommodated in the plurality of accommodating portions based on an input of a user; and
a processor configured to independently drive the plurality of illuminators so as to irradiate the items in the plurality of accommodating portions with the light based the dosage information corresponding to the type of each item selected by the input unit, based on a signal output by the door opening and closing sensor indicating that the door is closed and the temperature of the storage compartment measured by the temperature sensor being outside of a preset temperature range.

16. The refrigerator of claim 15, wherein the dosage information comprises an output (mW/cm²) per unit area and an irradiation time period of an LED light source of the plurality of illuminators based on the type of the item.

17. The refrigerator of claim 16, further comprising:
a distance sensor or a weight sensor configured to determine a distance between an item in an accommodating portion of the plurality of accommodating portions and the LED light source of a corresponding illuminator of the plurality of illuminators,
wherein the processor is further configured to control current applied to the corresponding illuminator based on the determined distance.

18. The refrigerator of claim 15, wherein the dosage information further comprises an irradiation time period for a preset dose based on a light output (mW/cm²) per unit area of an LED light source, and
wherein the processor is further configured to individually terminate driving of the plurality of illuminators, based on the irradiation time period elapsing.

19. The refrigerator of claim 18, wherein the processor is further configured to:
stop driving of the plurality of illuminators, based on the door being opened while driving the plurality of illuminators, and
drive the plurality of illuminators for a remaining irradiation time based on the door being closed after the door was opened.

20. A refrigerator comprising:
a body comprising a storage compartment in which a plurality of accommodating portions are provided;
a door provided on the body and configured to open and close the storage compartment;
a door opening/closing sensor configured to detect whether the door is open or closed;
a temperature sensor provided in the storage compartment, and configured to detect a temperature of the storage compartment;
a plurality of illuminators respectively provided in the plurality of accommodating portions, wherein each of the plurality of illuminators comprises a light-emitting diode (LED) light source configured to emit light having a wavelength of about 380 nm to about 420 nm, and the plurality of illuminators are configured to irradiate items accommodated in the plurality of accommodating portions with the light;
a distance measurement sensor or a weight sensor configured to determine a distance between an item in at least one of the plurality of accommodating portions and an LED light source of at least one of the plurality of illuminators;
a storage unit configured to store sterilization information comprising at least one preset dose for sterilization based on a type of the item; and
a processor configured to:
drive the at least one of the plurality of illuminators so as to irradiate the item accommodated in the at least one of the plurality of accommodating portions with the light at the preset dose, based on a signal output by the door opening and closing sensor indicating that the door is closed and the temperature of the storage compartment measured by the temperature sensor being outside of a preset temperature range,
control a current applied to the at least one of the plurality of illuminators, based on the determined distance, and
terminate driving of the at least one of the plurality of illuminators, based on an irradiation time period for the preset dose elapsing.
